(19) United States
(12) Patent Application Publication
(10) Pub. No.: US 2005/0101884 A1
(43) Pub. Date: May 12, 2005

(54) Apparatus and method for acquiring and processing data regarding physical attributes of a user

(75) Inventors: Tanya Ann Shaw Weeks, Windsor Junction (CA); Sheri Elizabeth McKillop, Dartmouth (CA); Joseph Rhoads Winston, Moorestown, NJ (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Unique Solutions Design Limited

(21) Appl. No.: 10/981,008

(22) Filed: Nov. 4, 2004

Related U.S. Application Data
(60) Provisional application No. 60/518,143, filed on Nov. 7, 2003.

(51) Int. Cl. A61B 5/103

(52) U.S. Cl. 600/587; 128/920; 600/300

(57) ABSTRACT

An apparatus for acquiring and processing data regarding physical attributes of a user includes a generally enclosed housing sized for receiving a user therein, the housing having an interior and an opening for entry into an exit from the housing by the user. A three-dimensional body scanner including a plurality of scanning heads is located within the housing for conducting a three-dimensional scan of the body of the user within the housing for obtaining accurate and repeatable spatial measurements of predetermined portions of the body of the user. A weighing device is provided for measuring the weight of the user. A data collection and processing device communicates with the body scanner and the weighing device for receiving, processing and analyzing data concerning the spatial measurements and weight of the user.

### CROSS-REFERENCE TO RELATED APPLICATIONS

This application claims the benefit of U.S. Provisional Patent Application No. 60/518,143 filed Nov. 7, 2003 and entitled “Method of Taking Human Body Measurements for the Health and Fitness Industries” the entire subject matter of which is incorporated herein by reference.

### BACKGROUND OF THE INVENTION

The present invention relates generally to a method and apparatus for acquiring and processing data regarding physical attributes of a user and, more particularly, to an automated method and apparatus for acquiring and processing such data in an accurate and repeatable manner.

In the fields of health and fitness, there are a number of methods which are currently used for helping users determine their state or status and progress in building muscle mass, losing weight and/or assessing overall health and fitness. The current methods include primarily manual measurement of the user's physical attributes such as height, weight, heart rate, body fat percentage and the size of waist, bust, abdomen, hips, thighs, calves, arms, etc. Convenience, accuracy and reliability are important to generate repeatable and accurate measurements in a manner that is acceptable to the user and provides an overall accurate assessment of health and fitness.

Such traditional approaches include the periodic taking of independent user measurements by manual means, including using tape measures, scales and other independent measuring devices. Typically such measurements are taken by a health or fitness staff member and are recorded for purposes of tracking the state of the health/fitness of the user and the user's progress. The measurements often vary in accuracy and repeatability due to variations in the techniques employed by the various different staff members. Accuracy may also be compromised by a staff member in an effort to encourage, persuade or assist the user in attaining a stated performance goal. The ability of the staff member to affect the measurement process in differing ways often leads to subjective, and not objective, data collection. While any such traditional measurements may each in their own turn be reliable, accurate and convenient, they do not constitute solutions which can be said to be at the same time reliable, repeatable, accurate and convenient.

The present invention provides a method and apparatus for obtaining multiple human body measurements using a system that integrates three dimensional imaging of the user's body with direct measurement of the weight and heart rate of the user. The measurements are all stored and can be used by the user to track his or her current health state as well as the progress of his or her health and fitness or can be sent to a health or fitness provider for the creation of specific assessments and plans. In the preferred embodiment, the present invention provides for a method of positively identifying the individual user at the time of the scanning and measurement process. In some embodiments, individual identification is performed using a bar code scanning device. In other embodiments, individual identification is performed using a text entry device or a graphical selection device. The present invention also provides a method for directing or assisting the individual user through the process of being scanned and measured. In some embodiments, speakers are used to provide audible cues to the user during the scanning and measurement process. In other embodiments, written and/or graphical instructions are located inside or outside of the scanning booth to direct the user through the process of being scanned and measured.

The present invention provides a method of taking a three-dimensional body scan of the individual user to obtain spatial measurements including, but not limited to, height, bust, waist, abdomen, hips, thighs, calves, arms and volume. Weight measurements of the user are also taken using a digital scale or other weighing device with the output of both the three-dimensional body scanner and the weighing device being connected to a data collection and computing device. Heart rate measurements of the user are also taken using electrodes and a signal processor with the output also being connected to the data collection and computing device. The present invention also provides a method of determining body fat percentage. In some embodiments, body fat percentage is computed utilizing the previously obtained volume and weight measurements. In other embodiments, body fat percentage is computed utilizing measured body impedance across electrodes with the output also being connected to the data collection and computing device.

In some embodiments of the invention, a network connection is provided to allow communication with a remote entity. Data compression techniques are used to minimize transfer time across the network connection. In some embodiments, measurements and/or measurement computations are performed locally and in other embodiments, the measurements and/or measurement computations are performed remotely.

The present invention also provides for a method of producing analytic data based upon the spatial measurements, weight measurements, body fat computations, heart rate measurements and other measurements. The produced analytic data may be specific to an individual user or to a class of individual users such as a group or all of the members of a particular health club, members at a particular health club location, members who have participated for a specified period of time, etc. The analytic data may also be produced utilizing historical data related to the individual user or class of individual users, aggregated data and/or other forms of computed and stored data. In some embodiments, the analytic data is produced locally and in other embodiments, the analytic data is produced remotely.

The present invention, in one embodiment, integrates measurements, computations and/or analytic data into a reliable, accurate and convenient assessment of the state and progress of the individual user's health and fitness. The measurements and/or analytic data may be transferred or displayed directly to the individual user and/or may be provided to the user's health and fitness club. Providing the measurements and/or analytic data to the user's health and fitness club, allows the club to assist the user in the interpretation of the measurements and/or analytic data. This also allows the club to develop specific health and fitness regimens with and for the individual user. In some embodiments, the measurements and/or analytic data are provided to the health and fitness club owner, franchisee or franchisor. This allows the owner, franchisee or franchisor to measure performance relative to other clubs or groups of clubs and to offer measurement and/or analytic data to users through a website or the like.

### BRIEF SUMMARY OF THE INVENTION

Briefly stated, in one embodiment, the present invention comprises an apparatus for acquiring and processing data regarding physical attributes of a user in an accurate, repeatable and automated manner. The apparatus comprises a generally enclosed housing sized for receiving a user therein. The housing has an interior and an opening for entry into and exit from the housing by the user. A three-dimensional body scanner, including a plurality of scanning heads, is located within the housing for conducting a three-dimensional body scan of a user within the housing for obtaining accurate spatial measurements of predetermined portions of the body of the user. A weighing device is provided for measuring the weight of the user. A data collection and processing device communicates with the body scanner and the weighing device for receiving, processing and analyzing data concerning the spatial measurements and weight of the user.

In another embodiment, the present invention comprises a method for acquiring and processing data regarding physical attributes of a user in an accurate, repeatable and automated manner. The method comprises the steps of: providing an enclosed housing for receiving a user therein; scanning the body of the user using a three dimensional scanner with a plurality of scanning heads; weighing the user using a weighing device; and receiving processing and analyzing data from the scanner and the weighing device by a data collection and processing device.

### BRIEF DESCRIPTION OF THE SEVERAL VIEWS OF THE DRAWINGS

The foregoing summary, as well as the following detailed description of the preferred embodiment of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a top plan schematic block diagram of a preferred embodiment of the present invention illustrating a housing and various components contained therein;

FIG. 2 is a schematic block diagram illustrating the communication paths between the various components of the apparatus shown in FIG. 1; and

FIG. 3 is a functional flow diagram of the principle steps involved in using a system of the type illustrated in FIG. 1.

### DETAILED DESCRIPTION OF THE INVENTION

Referring to the drawings in detail wherein the same numerals are used to indicate the same components throughout the several figures, there is shown in FIG. 1 a top plan schematic block diagram of an apparatus 10 for acquiring and processing data regarding physical attributes of a user. The apparatus 10 includes a housing 12 having an interior 14 and an opening 16 for entry into and exit from the housing by a user 18. The housing 12 may be a generally permanent structure or may comprise a more temporary structure such as a kiosk, booth, or the like. The housing 12 may be located within a health club, YMCA, gymnasium or other facility or, if desired, may comprise a stand alone structure, for example, within a mall or shopping center. Preferably, the opening 16 includes a door 20 which may be opened and closed by the user 18 during entry to and exit from the housing 12. The housing 12 could be a portable booth and could take on anyone of a variety of different shapes or dimensions if desired. The purpose of the housing 12 is to provide a facility for the below-described equipment and establishing an enclosed space to provide privacy for the user 18 when measurements are being taken. It will be appreciated by the those of ordinary skill in the art that the size and shape of the housing 12 as well as the location of the housing 12 and the materials from which the housing may be built will vary from application to application and should not be considered to be a limitation on the present invention. For example, in one embodiment, the housing 12 may comprise a series of curtains which define the interior 14 whereas in another embodiment, the housing 12 may comprise actual polymeric or steel walls.

In the present embodiment, the apparatus 10 includes an identification device for identifying the user 18. In the present embodiment, the identification device is a bar code reader 22 located within the interior 14 of the housing 12. Alternatively, the bar code reader 22 could be located outside of the housing 12, for example, proximate to the opening 16. As a further alternative, the identification device could comprise a device other than a bar code reader 22, for example, a text entry device (not shown), a graphical entry device (not shown), radio frequency identification device reader (not shown), finger print reader (not shown), or any other suitable identification device known to those of ordinary skill in the art. As shown in FIG. 1, the bar code reader 22 is positioned on a supporting surface 24 in one corner of the housing 22. However, the bar code reader 22 may be positioned at some other location, for example, next to the opening 16.

The apparatus 10 further includes a data collection and processing device, in the present embodiment a personal computer 26 which is also located on the supporting surface 24 in one corner of the housing 12. The bar code reader 22 is in communication with the data collection and processing device 26 so that when a bar code associated with the user 18 is read by the bar code reader 22, the read information is provided to the data collection and processing device 26 for comparison with stored data to provide confirmation of the identify of the user 18. While in the present embodiment the data collection and processing device 26 comprises a personal computer, it will appreciated by those of ordinary skill in the art that other suitable electronic equipment could alternatively be employed. In addition, while in the present embodiment the data collection and processing device 26 is located within the housing 12, the data processing and collection device 26 could be located outside of the housing 12 or, if desired, at a remote location.

The apparatus 10 further includes at least one speaker 28 which is also connected to the data collection and processing device 26. In the embodiment illustrated in FIG. 1, the speaker 28 is also located on the supporting surface 24 in one corner of the housing 12. However, it will be apparent to those of ordinary skill in the art that the speaker 28 could be positioned at some other location within the housing 12 if desired. The speaker 28 is employed for providing instructional information or audible clues to the user 18 to guide the user 18 through the scanning and measurement processes described below. More particularly, the audible instructions direct the user where to stand as certain measurements are being performed, prepares the user for the start and completion of the measurement scan and, once all of the scanning and measurement processes have been completed, inform the user that the measurement phase has been completed and invite the user to exit the housing 12. As an alternative to utilizing the speaker 28 and audible instructions, instructions for the measurement process may be accomplished either in written form or graphically, such as on a computer screen or liquid crystal display screen. Such instructions may be posted within the interior 14 of the housing 12 or may be posted on the outside of the housing 12 for viewing prior to the user 18 entering the housing 12. Other techniques or methods known to those of ordinary skill in the art may alternatively be employed for conveying instructions to the user 18.

The apparatus 10 includes a three dimensional body scanning system located within the housing 12 for conducting a three dimensional scan of the body of the user 18. The three dimensional body scan provides accurate spatial measurements of predetermined portions of the body of the user 18. In the embodiment shown in FIG. 1, the three dimensional body scanning system preferably includes four independent scanning heads 30, one of which is located on each of the walls of the housing 12. The scanning heads 30 are of a type well known to those of ordinary skill in the art and are employed to detect structured light reflected from the user 18 for the purpose of rendering a point cloud from which three dimensional body measurements can be accurately computed. The scanning heads 30 could be of the type that project and/or detect ambient light, sound, ultrasound, radar, microwave or any other suitable form or frequency of electromagnetic waves or other forms of measurement. It will appreciated that while four such scanning heads are provided in the presently preferred embodiment, a lesser or greater number of scanning heads could alternatively be provided. Further, while the four scanning heads 30 are located at spaced locations around the four walls of the housing 12, the scanning heads could be provided at other locations, for example, on three of the walls, or may be placed upon self-supporting stands at spaced locations within the interior 14 of the housing 12. It should therefore be clearly understood that the type, number and location of the scanning heads 30 should not be considered to be a limitation on the present invention. Each of the scanning heads 30 communicates with the data collection and processing device 26 to provide the scanned information or to facilitate the computation spatial measurements of predetermined portions of the body of the user 18. In the present embodiment, the computed spatial measurements include but are not limited to the user's height, bust size, waist size, abdomen size, hip size, thigh size, calf size, arm size, and volume. It will be appreciated by those of ordinary skill in the art that a lesser or greater number of spatial measurements may be computed, if desired, or that in some cases only limited spatial measurements may be computed. For example, if a user 18 is particularly interested in the development of one portion of his or her body, for example the arms, only the requisite arm measurements may be computed. Preferably, a user 18 is directed by the speaker 28 to stand at a particular location and assume a particular position for optimizing the operation of the scanning heads 30. Multiple user locations and/or positions may be assumed in order to obtain all of the necessary measurements.

The apparatus 10 also includes a weighing device fro measuring the weight of the user. In the preferred embodiment, the weighing device comprises a scale, preferably a digital scale 32 located within the interior of the housing 12. Alternatively, the weighing device could be an analog scale connected to an a/d converter or some other type of weighing device. Further, if desired, the weighing device or digital scale 32 could be located outside of the housing 12. The digital scale 32 is in communication with the data collection and processing device 26 for providing an indication of the weight of a user 18 for further processing and analysis as described below. In the preferred embodiment, a pair of handholds 34 are provided to assist the user 18 in connection with the use of the digital scale 32. The handholds 34 also provide stability to the user 18 during the scanning process. In the preferred embodiment, the handholds 34 include at least one and preferably two sensors (not shown) which are also in communication with the data collection and processing device 26. Each of the sensors determines the status of a predetermined functional condition of the body of the user 18 which in turn is communicated to the data collection and processing device 26. In the presently preferred embodiment, one of the sensors is a heart rate monitor such as the Insta-Pulse sensor from Biosig Instruments, Inc. Preferably, another sensor generates impedance measurements to permit body fat percentage to be calculated by the data collection and processing device 26. Alternatively, the body fat percentage may be measured with an independent sensing and measuring device also connected to the data connection and processing device 26. Further, the body fat percentage of the user 18 may be calculated through computations using the information obtained from the scanning heads 30 in combination of the information obtained from the digital scale 32. Other conditions of the body of the user 18 may be measured utilizing sensors located within the handholds or at other positions within the housing 12.

As previously mentioned, the data collection and processing device 26 is in communication with the barcode reader 22, speaker 28, scanning heads 30, digital scale 32 and the heart rate and body fat percentage sensors within the handholds 34. In this manner, the data collection and processing device 26 controls the barcode reader 22 or other identification device and outputs audio signals to the speakers 28 to thereby provide instructions to the user 18. The data collection and processing 26 controls the operation of the scanning heads 30 and receives and records the all of the scanned data. The data collection of processing device 26 also controls the digital scale 32 and receives the data from the digital scale 32 as well as the from the heart rate and body fat percentage sensors within the handholds 34. In the preferred embodiment, the data collection processing device 26 compresses all of the data obtained from the barcode reader 22, scanning heads 30, digital scale 32 and the sensors within the handholds 34 utilizing compression techniques well known to those of ordinary skill in the art. The compressed data is then sent by the data collection and processing device 26 to a remote entity (not shown) for further processing as described below. The connection between the data collection and processing device 26 and the remote entity may be made over a dialup connection through a telephone line 32 or through a broadband connection over cable line 38.

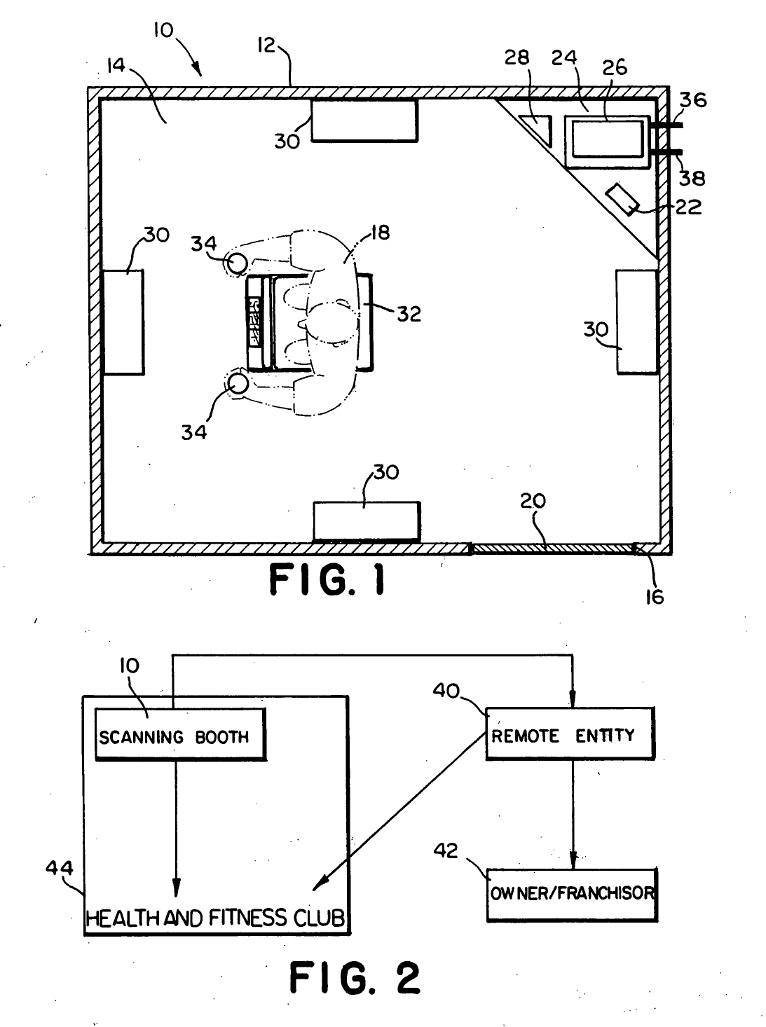

FIG. 2. shows the communications extending between the apparatus 10 as shown in FIG. 1 and three other remote systems including a remote entity 40 (hereinafter discussed) an owner/franchisor entity 42 and a health and fitness club 44 within which the apparatus 10 is located. In each case, the communication extends from the data collection and processing device 26 to a similar or related computer device such as a personal computer or server located in the remote entity owner/franchisor 42 or health and fitness club 44. The data transferred from the data collection and processing device 26 may or may not be compressed depending upon the system used for transferring the data. For example, if a dialup system is used the data will be compressed whereas if a broadband system is used compression may not be necessary. In the present embodiment, the remote entity 40 includes a server (not shown) which incorporates a database. Both the server and the database are of a type well known for those of ordinary skill in the art. The server and database at the remote entity 40 perform additional processing on the collected data, performs analysis on current, historical, individual and aggregated data and integrates the results into reports for the individual user 18, for the health and fitness club 44 and, in some cases for the owner/franchisor 42. In the preferred embodiment, reports for the user 18 and the health and fitness club 44 are both sent to the health and fitness club 44. In this manner, a staff member of the health and fitness club 44 may assist the user in the interpretation of the measurements and/or analytic data produced at the remote entity server. In this manner, the staff members of the health club may develop health and fitness regiments which are specifically tailored to each individual user 18. Alternatively, the reports may be sent by fax or by regular mail to the health and fitness club 44. In the preferred embodiment of the invention, the reports are also sent by email to the owner/franchisor 42. The owner/franchisor 42 may use the reports and, particularly, the analytic data within the reports to assess the overall performance of individual health and fitness clubs or groups of health and fitness clubs against aggregated data. In this manner, the owner/franchisor may offer measurement and/or analytic data to the user to a branded website to add value to health and fitness club membership. Alternatively, the reports may be sent to the owner/franchisor 42 by fax or by regular mail. As a further alternative, the owner/franchisor may access the reports from the server at the remote entity 40 by using the internet. In an alternate embodiment, all of the data processing and analysis performed by the server at the remote entity 40 may be performed by utilizing the data collection and processing device 26 within the apparatus 10. In this embodiment, reports containing the integrated measurement and analytic data may be sent by email, fax or mail to the health and fitness club and/or to the owner/franchisor.

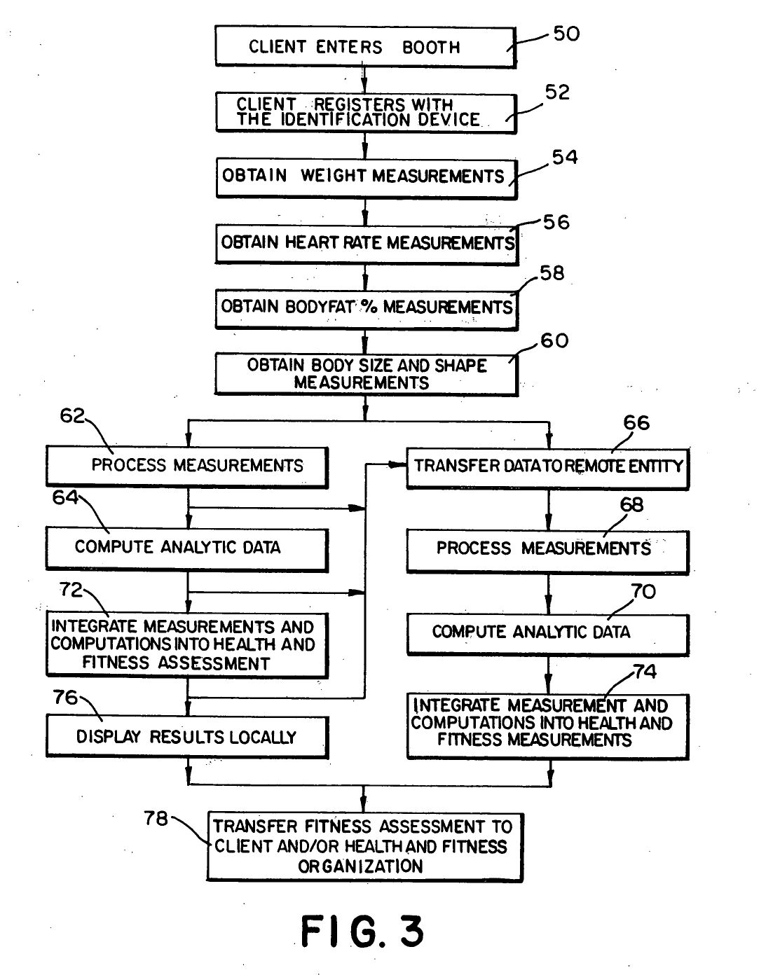

FIG. 3 is a functional flow diagram of the principal steps in utilizing the apparatus 10 in accordance with the present invention. In the first step 50, the client or user 18 enters the booth or housing 12 through the door 20. In the next step 52, the client or user 18 registers with the identification device, preferably by a scanning a card, wristband or the like containing a barcode utilizing the barcode reader 22. In step 54, the user steps onto the weighing device or digital scale 32 for obtaining his or her weight measurements. While the weight measurements are being obtained, the user 18 grasps the handholds 34 and his or her heartrate measurements and bodyfat measurements are obtained utilizing the sensors in the handholds 34 as shown in steps 56 and 58. Simultaneously or shortly thereafter, the scanning heads 30 are activated to obtain the body size and shape measurements of the user 18 as shown in step 60. All of the measurements of steps 54-60 are taken under the control of the data collection and processing device 26 and the readings obtained are communicated to and stored within the data collection and processing device 26. At step 62, the data collection and processing device 26 processes the measurement obtained from the user in the foregoing steps and at step 64 the data collection and processing device 26 computes the analytic data utilizing software and store information. Alternatively, or concurrently, the data obtained from the user are transferred to the server within the remote entity 40 in step 66 and in steps 68 and 70 the server at the remote entity processes the data and computes the analytic data based upon software and previously stored data. Regardless of where the data analysis takes place, the resulting measurements and analysis are integrated into health and fitness assessments at steps 72 and 74. If the health and fitness assessment is obtained locally (i.e., near the apparatus 10), the results may be displayed to the user 18 at step 76. In either event, the health and fitness assessment is thereafter transferred to the user, and/or the health and fitness club 44 and/or the owner/franchisee of the health and fitness club 42 for further analysis, processing and use as described above in step 78.

From the foregoing, it can be seen that the present invention comprises a method and apparatus for acquiring and processing data regarding the physical attributes of a user in an automated accurate and repeatable manner. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An apparatus for acquiring and processing data regarding physical attributes of a user in an accurate, repeatable and automated manner comprising:
a generally enclosed housing sized for receiving a user therein, the housing having an interior and an opening for entry into and exit from the housing by the user;
a three dimensional body scanner including a plurality of scanning heads located within the housing for conducting a three dimensional scan of the body of a user within the housing for obtaining accurate spatial measurements of predetermined portions of the body of the user;
a weighing device for measuring the weight of the user; and
a data collection and processing device communicating with the body scanner, and the weighing device for receiving, processing and analyzing data concerning the spatial measurements and weight of the user.

2. The apparatus as recited in claim 1 wherein the housing opening includes a door.

3. The apparatus as recited in claim 1 further including an identification device for identifying the user, the identification device also communicating with the data collection device.

4. The apparatus as recited in claim 3 wherein the identification device is located within the housing.

5. The apparatus as recited in claim 3 wherein the identification device comprised one of a bar code reader, a text entry device and a graphical entry device.

6. The apparatus as recited in claim 1 wherein the scanning heads project and/or detect at least one of light, sound, ultra-sound, radar, microwave and other electromagnetic frequencies.

7. The apparatus as recited in claim 1 wherein the predetermined portions of the body for which the spatial measurements are obtained include at least one of height, bust size, waist size, abdomen size, hips size, thigh size, calve size arm size and volume.

8. The apparatus as recited in claim 1 wherein the weighing device comprises a scale located within the interior of the housing.

9. The apparatus as recited in claim 8 wherein the scale comprises one of a digital scale and an analog scale with an analog to digital converter.

10. The apparatus as recited in claim 1 further including a sensor for determining the status of a predetermined functional condition of the body of the user, the sensor also communicating with the data collection device to provide information regarding the status of the functional condition.

11. The apparatus as recited in claim 1 wherein the sensor measures at least one of heart rate and body fat percentage.

12. The apparatus as recited in claim 1 further including means for analyzing the spatial measurements and weight of the user to produce a health and fitness regiment for the user.

13. The apparatus as recited in claim 1 further including means for analyzing the spatial measurements and weight of the multiple users for assessing the performance of a health and fitness facility.

14. A method of acquiring and processing data regarding physical attributes of a user comprising the steps of:
providing and enclosed housing for receiving a user therein;
scanning the body of the user within the housing using a three-dimensional body scanner including a plurality of scanning heads;
weighing the user using a weighing device; and
receiving, processing and analyzing data from the scanner and the weighing device by a data collection and processing device.

15. The method as recited in claim 14 further comprising the steps of identifying the user using an identifying device communicating with the data collection and processing device.

16. The method as recited in claim 14 further including the step of measuring the heart rate of the user and communicating the measured heart rate to the data collection and processing device.

17. The method as recited in claim 14 further including the step of measuring the body fat percentage of the user and communicating the measured body fat percentage to the data collection and processing device.

18. The method as recited in claim 14 further including the step of analyzing the data from the scanner and the weighing device to produce a health and fitness regimen for the user.
